(19) United States
(12) Patent Application Publication
(10) Pub. No.: US 2020/0205901 A1
(43) Pub. Date: Jul. 2, 2020

(54) INSTRUMENT PATH GUIDANCE USING VISUALIZATION AND FLUORESCENCE

(71) Applicant: TransEnterix Surgical, Inc., Morrisville, NC (US)

(72) Inventors: Matthew Robert Penny, Holly Springs, NC (US); Kevin Andrew Hufford, Cary, NC (US); Mohan Nathan, Raleigh, NC (US); Glenn Warren, Raleigh, NC (US)

(21) Appl. No.: 16/732,304

(22) Filed: Dec. 31, 2019

Related U.S. Application Data
(60) Provisional application No. 62/787,250, filed on Dec. 31, 2018.

(51) Int. Cl. A61B 34/10; A61B 34/37; A61B 34/00; A61B 18/00

(52) U.S. Cl. CPC A61B 34/10; A61B 34/37; A61B 2034/302; A61B 18/00; A61B 34/76

(57) ABSTRACT

In a surgical method using a robotic system, a distal end of a robotically controlled surgical instrument is positioned in a patient body cavity. Operation of the instrument is controlled in response to input provided by a surgeon at an input device. An image of the interior of the body cavity is captured for display on a display. A boundary in the body cavity is identified using the image processing software by distinguishing between different colors on the image. In response to identification of the boundary, at least one of the following modes of operation is performed:
providing a haptic contract at the surgeon console constraining movement of the surgical instrument to maintain the instrument along the boundary;
preventing activation of an electrosurgical function of the instrument except with the instrument is within a defined distance from the boundary;
allowing activation of an electrosurgical function of the instrument only when the instrument is positioned along the boundary

### BACKGROUND

Surgical robots enable enhanced control of instruments via basic features such as tremor-filtration and motion scaling. When paired with advanced vision systems, new opportunities for surgical instrument control arise. Current implementations of vision systems such as MRI and surgical robots like the Mako™ robot from Stryker enable paths and boundaries in orthopedic procedures and other hard tissue interventions.

Although the concepts described herein may be used on a variety of robotic surgical systems, one robotic surgical system is shown in FIG. 1. In the illustrated system, a surgeon console 12 has two input devices such as handles 17, 18. The input devices 12 are configured to be manipulated by a user to generate signals that are used to command motion of a robotically controlled device in multiple degrees of freedom. In use, the user selectively assigns the two handles 17, 18 to two of the robotic manipulators 13, 14, 15, allowing surgeon control of two of the surgical instruments 10a, 10b, and 10c disposed at the working site (in a patient on patient bed 2) at any given time. To control a third one of the instruments disposed at the working site, one of the two handles 17, 18 may be operatively disengaged from one of the initial two instruments and then operatively paired with the third instrument, or another form of input may control the third instrument as described in the next paragraph. A fourth robotic manipulator, not shown in FIG. 1, may be optionally provided to support and maneuver an additional instrument.

One of the instruments 10a, 10b, 10c is a camera that captures images of the operative field in the body cavity. The camera may be moved by its corresponding robotic manipulator using input from a variety of types of input devices, including, without limitation, one of the handles 17, 18, additional controls on the console, a foot pedal, an eye tracker 21, voice controller, etc. The console may also include a display or monitor 23 configured to display the images captured by the camera, and for optionally displaying system information, patient information, etc.

A control unit 30 is operationally connected to the robotic arms and to the user interface. The control unit receives user input from the input devices corresponding to the desired movement of the surgical instruments, and the robotic arms are caused to manipulate the surgical instruments accordingly.

The input devices 17, 18 are configured to be manipulated by a user to generate signals that are processed by the system to generate instructions used to command motion of the manipulators in order to move the instruments in multiple degrees of freedom and to, as appropriate, control operation of electromechanical actuators/motors that drive motion and/or actuation of the instrument end effectors.

The surgical system allows the operating room staff to remove and replace the surgical instruments 10a, b, c carried by the robotic manipulator, based on the surgical need. When an instrument exchange is necessary, surgical personnel remove an instrument from a manipulator arm and replace it with another.

This application details embodiments where the vision system used intraoperatively with the surgical robot is able to distinguish and identify tissue planes, paths, anatomical landmarks and structures and the surgical robot uses that information to enable advanced control of surgical instruments. The embodiments herein will focus on soft tissue interventions but could have applicability in other dynamic scenarios as well.

### BRIEF DESCRIPTION OF THE DRAWINGS

FIG. 1 shows an exemplary surgical robot system with which the concepts described herein may be used.

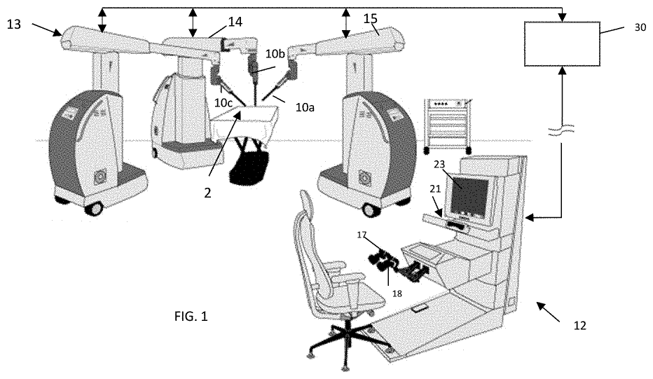

FIG. 2 shows a screen capture of an image of a surgical site captured using a laparoscopic camera.

FIG. 3 shows a screen capture of an image of a surgical site as displayed on a display, and further shows an overlay generated on the display.

### DETAILED DESCRIPTION

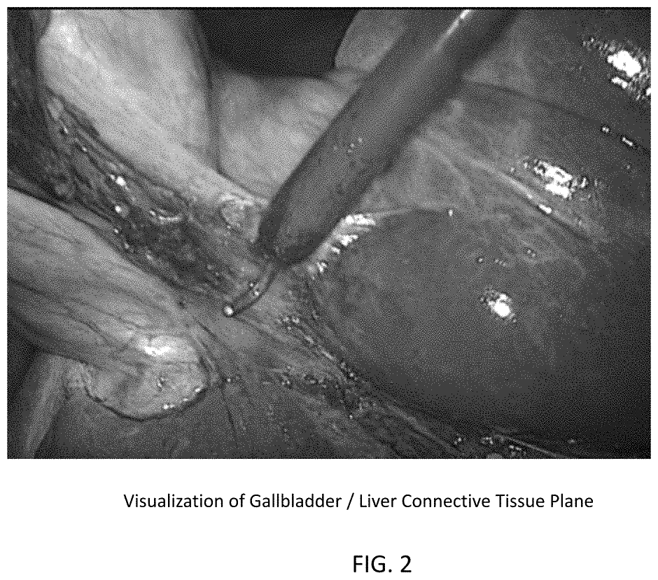

A first embodiment pairs a surgical robot system with a standard, off-the-shelf visualization system (such as Stryker or Olympus, etc.). The image processing equipment is designed to enable differentiation of objects within the field of view based on the RGB values. As an example, the FIG. 2 image is a screen capture from a laparoscopic cholecystectomy. Note the difference in the coloration of the liver tissue (bottom right) and the gallbladder tissue (top left). In accordance with one aspect of the present invention, the image processing equipment distinguishes between these two colors and defines an intersection path between the two tissues. The surgical system could use this intersection path for a multitude of features and operative modes. For example, in one mode of operation the monopolar hook could be haptically constrained with the tip at the intersection between the two tissues. This haptic constraint would help the surgeon to apply monopolar energy at exactly the right tissue plane, preventing gallbladder puncture or liver damage from electrocautery. The haptic constraint could act like a magnet—only exerting force when the instrument gets close to the defined path or object. This would allow the surgeon to freely move about the surgical field but feel the path or object when he is close.

A second mode would restrict use of an electrosurgical device, by preventing monopolar energy from being activated from the instrument unless the instrument was positioned to deliver that energy within a region bordering the identified intersection between the two tissues. This could also prevent undesired damage to adjacent tissue.

A third operative mode may enable boundaries based on tissue color identification. These boundaries may either keep surgical instruments within a given area (keep-in), or outside of a given area (keep-out). The surgical system would haptically prevent the user from moving instruments out of/into those regions, as applicable.

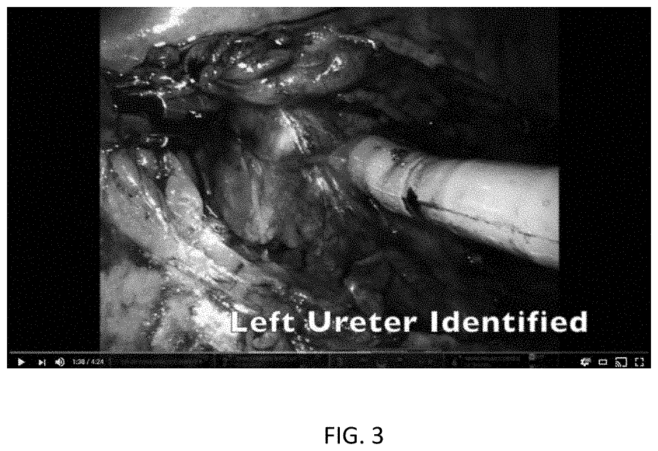

A second embodiment would enable the use of a surgical robotic system with an advanced visualization system (such as the one from Novadaq) equipped with fluorescence imaging technology. See the image below which illustrates a hidden structure that has been illuminated using a fluorescing dye, placed prior to the surgical intervention in the structure or blood stream. The image processing equipment would identify the presence of fluorescence and use the differentiation between the fluorescing object and surrounding tissue to identify paths or boundaries for the surgical robot. See FIG. 3. The modes of operation could be similar to those described in the primary embodiment.

The disclosed concepts are advantageous in that they define a path or region definition using visualization and tissue differentiation based on color or fluorescence. Operative modes for a surgical robot use the on paths or regions defined with the image processing equipment to, for example, prevent or allow certain types of activity, in some cases allowing the user to “feel” identified boundaries or paths via haptic constraints, attractions or repulsions. In some cases those modes that enable the use of instrument features the instrument is when near identified paths, objects or boundaries. Others disable the use of instrument features when the instrument is near identified paths, objects or boundaries.

It will be appreciated that the concepts described here may be used in conjunction with systems and modes of operation described in co-pending U.S. application Ser. No. 16/237,444, entitled “System and Method for Controlling a Robotic Surgical System Based on Identified Structures” which is incorporated herein by reference.

We claim:

1. A surgical method, comprising:
providing a robotic manipulator and a surgical instrument removably attached to the robotic manipulator,
positioning a distal end of the surgical instrument in a patient body cavity and controlling operation of the instrument by providing input at a surgeon console;
capturing an image of the body cavity for display on a display;
identifying a boundary in the body cavity using the image processing software by distinguishing between different colors on the image;
in response to identification of the boundary, performing at least one of the following:
providing a haptic contract at the surgeon console constraining movement of the surgical instrument to maintain the instrument along the boundary;
preventing activation of an electrosurgical function of the instrument except with the instrument is within a defined distance from the boundary;
allowing activation of an electrosurgical function of the instrument only when the instrument is positioned along the boundary

2. The method of claim 1, where the image processing software uses the color or fluorescence of tissues within the operative view to define paths, objects or boundaries.
